(19) United States
(12) Patent Application Publication
(10) Pub. No.: US 2018/0369307 A1
(43) Pub. Date: Dec. 27, 2018

(54) HERBAL FORMULATION FOR REGENERATING CARDIOMYOCYTES BY REDUCING THE AFTER EFFECTS OF MYOCARDIAL INFARCTION

(71) Applicant: Ravishankar POLISETTY, Mississauga (CA)

(72) Inventor: Ravishankar POLISETTY, Mississauga (CA)

(21) Appl. No.: 15/630,004

(22) Filed: Jun. 22, 2017

(51) Int. Cl. A61K 36/185; A61K 36/84

(52) U.S. Cl. CPC A61K 36/185; A61K 36/84

(57) ABSTRACT

An herbal formulation employing a plurality of ingredients extracted from a plurality of herbs for the regeneration of cardiomyocytes by reducing the after effects of myocardial infarction is disclosed. the herbal formulation made from one or all of the ingredients include Terminalia arjuna, Pashanbhedi (Bergenia lingulata), Jatamansi (Nardostachys jatamansi), Swarnabhasma (gold ultra fine powder), Gandhakrasayan, Trifala (Myrabilis), Commiphoramukul, Triphala (three myrobalans) andcalcined pearl shell powder (Mukthapisthi) in appropriate percentage by weight of the composition. According to the method of preparing the herbal formulation, the ingredients are purified by liquid solvent extraction and homogenization of herbal ingredients is achieved to attain a homogenous formulation. The formulation is prepared in any of the forms include but not limited to a gelatin capsule, a vegetarian capsule, a tablet, a liquid, a syrup, a dairy beverage and a snack bar.

### TECHNICAL FIELD OF THE INVENTION

The present invention relates to an herbal composition for regenerating cardiomyocytes. More particularly, the present invention relates to a method for preparing an herbal formulation employing a plurality of ingredients extracted from a plurality of herbs for the protection of cardiomyocytes by reducing the after effects of myocardial infarction.

### BACKGROUND OF THE INVENTION

Heart is a vital organ in the human body responsible for pumping blood throughout the blood vessels. Diseases affecting the heart may be structural or functional. Anything which damages heart or decreases heart's supply of oxygen, makes it less efficient, reduces its ability to fill and pump, disrupts a coordinated relationship between the heart, kidneys, and blood vessels and harms not only the heart but the rest of the body as well.

Recently in developing countries, most of the people are affected by ischemic heart disease and myocardial infarction and is becoming a major cause of death. The coronary arteries deliver oxygenated blood to the heart muscles. If one of the arteries blocked suddenly the portion of heart is starved of oxygen and heart tissues die. The main reason for development of myocardial infarction is thrombosis i.e. clotting of blood in blood vessel and occlusion i.e. blockage of coronary vessels which entails a disturbance or stagnation of local blood circulation and the development of necrosis of cardiomyocytes.

To prevent myocardial infarction through multiple mechanism underlying the pathogenesis of this disease. One method of prevention is associated with correction of disorders of cholesterol metabolism inhibition of cholesterol synthesis, reducing its absorption in intestines, the exception from the diet of products rich in cholesterol and accelerating the excretion of cholesterol. Other ways include prevention of stress-induced myocardial damage and reducing adverse changes in metabolism caused by a stress situation. There is a possibility of regeneration of myocardial through resident stem cells of the myocardium as well as hematopoietic and mesenchymal stem cells. Another way of myocardial recovery growth through the exposure of macrophages that is capable of producing a number of necessary growth regulators to immune modulators.

There is a wide range of medicinal herbs which are aimed at the abovementioned mechanisms for the prevention of ischemic heart disease and myocardial infarction which reduces blood clotting and preventing thrombosis.

Therefore, there is a need to develop an herbal formulation that have a vital effect on the pathogenesis of ischemic heart disease and myocardial infarction that decreases the risk of disease and reduces the impact of it.

### BRIEF SUMMARY OF THE INVENTION

The present invention recognizes the limitations of the prior art and the need for systems and methods that are able to provide assistance to users in a manner that overcomes these limitations.

A principal object of the present invention is to an herbal formulation that shows a vital effect on the pathogenesis of ischemic heart disease and myocardial infarction by reducing the risk of disease and its impact.

Another object of the present invention is to provide a plurality of ingredients of the herbal formulation for reducing the after effects of myocardial infarction.

Yet Another object of the present invention is to prepare the formulation in any of the forms include but not limited to a gelatin capsule, a vegetarian capsule, a tablet, a liquid, a syrup, a dairy beverageand a snack bar.

According to a first aspect of the present invention, a herbal formulation for reducing the after effects of myocardial infarction to protect cardiomyocytes which ultimately shortens the risk of disease and its impact comprising a terminalia arjuna in an amount of 2-90% by weight of the composition, a pashanbhedi (bergenia lingulata) in an amount of 2-90% by weight of the composition, ajatamansi (nardostachys jatamansi) in an amount of 2-90% by weight of the composition, a swarnabhasma (gold ultra fine powder) in an amount of 2 ppm-6 ppm by weight of the composition, a gandhakrasayan in an amount of 2 ppm-6 ppm by weight of the composition, a trifala (three myrobalans) in an amount of 2-90% by weight of the composition, a commiphoramukul in an amount of 2-90% by weight of the composition and a calcined pearl shell powder (mukthapisthi) in an amount of 1.25-10% by weight of the composition.

In accordance with a first aspect of the present invention, a herbal formulation wherein more specifically comprises of a terminalia arjuna in an amount of 33.3% by weight of the composition, a pashanbhedi (bergenia lingulata) in an amount of 16.7% by weight of the composition, a jatamansi (nardostachys jatamansi) in an amount of 10% by weight of the composition, a swarnabhasma (gold ultra fine powder) in an amount of 6 ppm by weight of the composition, a gandhakrasayan in an amount of 2 ppm by weight of the composition, a trifala (three myrobalans) in an amount of 23.3% by weight of the composition, a commiphoramukul in an amount of 15.4% by weight of the composition and a calcined pearl shell powder (mukthapisthi) in an amount of 1.25% by weight of the composition.

In accordance with a first aspect of the present invention, further the herbal formulation is prepared in at least one of the forms include but not limited to a gelatin capsule, a vegetarian capsule, a tablet, a liquid, syrup, a dry beverage and a snack bar.

In accordance with a first aspect of the present invention, a herbal formulation can also be used in various forms comprising an atherosclerotic plaque reducer, as an anti-oxidant, as an anti-inflammatory agent, as an anxiolytic, as a cardioprotective agent or as a cardiac tonic.

In accordance with a first aspect of the present invention, a herbal formulation is used for the treatment of cardiac related problems include myocardial infarction, stroke, ischemia, intermittent claudication, post-MI scar, and for the regeneration of tissue destroyed as a result of MI.

In accordance with a first aspect of the present invention, the herbal formulation consisting of a various components includes a mixture of active ingredients extracted from the herbs.

According to a second aspect of the present invention, a method for preparing an herbal formulation for reducing the after effects of myocardial infarction to protect cardiomyocytes which ultimately shortens the risk of disease and its impact. The method further comprises steps of grinding the herbal ingredients forming a formulation to form a fine powder, purifying the herbal ingredients through a plurality of ways to prepare an effective formulation, homogenization of herbal ingredients to attain a homogenous preparation, mixing the herbal ingredients according to the weight of the composition of the components and processing the mixed formulation into an acceptable form of administration.

In accordance with a second aspect of the present invention, wherein purifying of the herbal ingredients through one or more methods include a liquid solvent extraction, a 0.01% phthalazine solution processing followed by extraction, a 0.02-2% urea processing followed by extraction, a chlorofluorocarbon gas extraction and carbon dioxide extraction.

In accordance with a second aspect of the present invention, further the method of processing herbal mixture into acceptable form include a gelatin capsule, a vegetarian capsule, a tablet, a liquid, a syrup, a dairy beverage and a snack bar.

In accordance with a second aspect of the present invention, wherein the method of preparing a herbal formulation of the composition can also be used in various forms comprising an atherosclerotic plaque reducer, as an anti-oxidant, as an anti-inflammatory agent, as an anxiolytic, as a cardioprotective agent, as a cardiac tonic for the treatment of cardiac related problems include myocardial infarction, stroke, ischemia, intermittent claudication, post-MI scar and for the regeneration of tissue destroyed as a result of MI.

### BRIEF DESCRIPTION OF THE DRAWINGS

Other objects and advantages of the present invention will become apparent to those skilled in the art upon reading the following detailed description of the preferred embodiments, in conjunction with the accompanying drawings, wherein like reference numerals have been used to designate like elements, and wherein FIG. 1 illustrates a process flow chart depicting the step-by-step process of the preparation of the herbal formulation according to the present invention.

FIGS. 2-5 are exemplary illustrations of the infarction area on the 1^{st}, 7^{th}, 14^{th} & 14^{th} days respectively without administration of the herbal formulation according to the present invention.

FIG. 6 is an exemplary illustration of the necrosis area on the 1^{st} day after the operation with herbal formulation according to the present invention.

FIGS. 7-8 are exemplary illustrations of the infraction area on 1^{st} and 5^{th} days after the operation with herbal formulation according to the present invention.

FIGS. 9a-9c are exemplary illustrations of the sub epicardial area of myocardial necrosis, infraction area and thymus on the 7^{th}, 14^{th} and 1^{st} day respectively of the operation with herbal formulation according to present invention.

FIG. 10a is an exemplary illustration of infraction area on the 14^{th} day after the operation with preliminary herbal treatment according to present invention.

FIG. 10b is an exemplary illustration of the thymus on the 5^{th} day after the operation and administration of herbal formulation according to present invention.

FIGS. 11-12 are exemplary illustrations of the liver on the 1^{st} and 5^{th} day after the operation and administration of herbal formulation according to the present invention.

FIGS. 13-14 are exemplary illustrations of the spleen on the 1^{st} and 5^{th} day after the operation and administration of herbal formulation according to the present invention.

FIGS. 15-16 are exemplary illustrations of the kidney on the 1^{st} and 5^{th} day after the operation and administration of herbal formulation according to the present invention.

### DETAILED DESCRIPTION OF THE INVENTION

The present invention is directed towards an herbal formulation formed from various herbs for reducing the after effects of myocardial infarction for regenerating cardiomyocytes. Referring to the drawings, designate the embodiments of the results before and after the treatment of a person with the herbal formulation of the effected body parts due to myocardial infarction of the present invention are described.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced item. Further, the use of terms “first”, “second”, and “third”, and the like, herein do not denote any order, quantity, or importance, but rather are used to distinguish one element from another.

In accordance to an exemplary embodiment of the present invention, a herbal formulation for reducing the after effects of myocardial infarction to protect cardiomyocytes which ultimately shortens the risk of disease and its impact comprising a terminalia arjuna in an amount of 2-90% by weight of the composition, a pashanbhedi (bergenia lingulata) in an amount of 2-90% by weight of the composition, a jatamansi (nardostachys jatamansi) in an amount of 2-90% by weight of the composition, a swarnabhasma (gold ultra fine powder) in an amount of 2 ppm-6 ppm by weight of the composition, a gandhakrasayan in an amount of 2 ppm-6 ppm by weight of the composition, a trifala (three myrobalans) in an amount of 2-90% by weight of the composition, a commiphoramukul in an amount of 2-90% by weight of the composition and a calcined pearl shell powder (mukthapisthi) in an amount of 1.25-10% by weight of the composition.

In accordance to an exemplary embodiment of the present invention, a herbal formulation wherein more specifically comprises of a terminalia arjuna in an amount of 33.3% by weight of the composition, a pashanbhedi (bergenia lingulata) in an amount of 16.7% by weight of the composition, a jatamansi(nardostachys jatamansi) in an amount of 10% by weight of the composition, a swamabhasma (gold ultra fine powder) in an amount of 6 ppm by weight of the composition, a gandhakrasayan in an amount of 2 ppm by weight of the composition, a trifala (three myrobalans) in an amount of 23.3% by weight of the composition, a commiphoramukul in an amount of 15.4% by weight of the composition and a calcined pearl shell powder (mukthapisthi) in an amount of 1.25% by weight of the composition.

In accordance to an exemplary embodiment of the present invention, further the herbal formulation is prepared in at least one of the forms include but not limited to a gelatin capsule, a vegetarian capsule, a tablet, a liquid, syrup, a dry beverage and a snack bar.

In accordance to an exemplary embodiment of the present invention, an herbal formulation can also be used in various forms comprising an atherosclerotic plaque reducer, as an anti-oxidant, as an anti-inflammatory agent, as an anxiolytic, as a cardioprotective agent or as a cardiac tonic.

In accordance to an exemplary embodiment of the present invention, a herbal formulation is used for the treatment of cardiac related problems include myocardial infarction, stroke, ischemia, intermittent claudication, post-MI scar, and for the regeneration of tissue destroyed as a result of MI.

In accordance to an exemplary embodiment of the present invention, the herbal formulation consisting of a various components includes a mixture of active ingredients extracted from the herbs.

In accordance to an exemplary embodiment of the present invention, a method for preparing a herbal formulation for reducing the after effects of myocardial infarction to protect cardiomyocytes which ultimately shortens the risk of disease and its impact. The method further comprises steps of grinding the herbal ingredients forming a formulation to form a fine powder, purifying the herbal ingredients through a plurality of ways to prepare an effective formulation, homogenization of herbal ingredients to attain a homogenous preparation, mixing the herbal ingredients according to the weight of the composition of the components and processing the mixed formulation into an acceptable form of administration.

In accordance to an exemplary embodiment of the present invention, wherein purifying of the herbal ingredients through one or more methods include a liquid solvent extraction, a 0.01% phthalazine solution processing followed by extraction, a 0.02-2% urea processing followed by extraction, a chlorofluorocarbon gas extraction and carbon dioxide extraction.

In accordance to an exemplary embodiment of the present invention, further the method of processing herbal mixture into acceptable form include a gelatin capsule, a vegetarian capsule, a tablet, a liquid, a syrup, a dairy beverage and a snack bar.

In accordance to an exemplary embodiment of the present invention, wherein the method of preparing a herbal formulation of the composition can also be used in various forms comprising an atherosclerotic plaque reducer, as an anti-oxidant, as an anti-inflammatory agent, as an anxiolytic, as a cardioprotective agent, as a cardiac tonic for the treatment of cardiac related problems include myocardial infarction, stroke, ischemia, intermittent claudication, post-MI scar and for the regeneration of tissue destroyed as a result of MI.

Referring to drawings illustrating the results before and after the treatment of a person effected with myocardial infarction with the herbal formulation of the present invention are described.

FIG. 1 illustrates a process flow chart 100 depicting the step-by step process of the preparation of the herbal formulation. At the start of the process 102, the herbal composition according to the present invention is made from a plurality of herbal components 104. These components include but not limited to Terminalia arjuna, Pashanbhedi (Bergenia lingulata), Jatamansi (Nardostachys jatamansi), Swarnabhasma (gold ultra fine powder), Gandhakrasayan, Trifala (Myrabilis), Commiphoramukul, Triphala (three myrobalans) andcalcined pearl shell powder (Mukthapisthi) or the like. At the next step of process 106, each herbal composition is considered as per the weight percentage of the composition include 2-90% is the weight composition of Terminalia arjuna, 2-90% is the weight composition of Pashanbhedi (Bergenia lingulata), 2-90% is the weight composition of Jatamansi (Nardostachys jatamansi), 2 ppm-6 ppm is the weight composition of Swarnabhasma (gold ultra fine powder), 2 ppm-6 ppm is the weight composition of Gandhakrasayan, 2-90% is the weight composition of Trifala (three myrobalans), 2-90% is the weight composition of Commiphoramukuland 1.25%-10% is the weight composition of calcined pearl shell powder (Mukthapisthi).

Even more specifically the herbal formulation comprises ingredients include but not limited to a 33.3% by weight composition is the Terminalia arjuna, 16.7% by weight of the composition is the Pashanbhedi (Bergenia lingulata), 10% by weight of the composition is the Jatamansi (Nardostachys jatamansi), a 6 ppm by weight of the composition is the Swarnabhasma (gold ultra fine powder), a 2 ppm by weight of the composition is the Gandhakrasayan, 23.3% by weight of the composition is the Trifala (Myrabilis), 15.4% by weight of the composition is the Commiphoramukul, and 1.25% by weight of the composition is the calcined pearl shell powder (Mukthapisthi).

At further step 108, the herbal ingredients are grinded to form a fine powder and later at step 110 purify the herbal ingredients through a plurality of ways listed at step 112 include but not limited to a liquid solvent extraction, a 0.01% phthalazine solution processing followed by extraction, a 0.02-2% urea processing followed by extraction, a chlorofluorocarbon gas extraction and a carbon dioxide extraction. At step 114, the homogenization of the herbal ingredients is attained to make the preparation a homogenous formulation. Further at step 116, the homogenized ingredients are mixed as per the weight composition of the preparation.

At final step 118, the herbal formulation mixture is processed into an acceptable form consisting of a gelatin capsule, a vegetarian capsule, a tablet, a liquid, syrup, a dairy beverage, a snack bar or the like and ends the process at 120. This herbal formulation is also used in various forms comprising an atherosclerotic plaque reducer, as an anti-oxidant, as an anti-inflammatory agent, as an anxiolytic, as a cardioprotective agent, as a cardiac tonic for the treatment of cardiac related problems include myocardial infarction, stroke, ischemia, intermittent claudication, post-MI scar and for the regeneration of tissue destroyed as a result of MI.

Use of this herbal formulation for treating above listed problems is possible, since the stroke and intermittent claudication are closely associated with the development of atherosclerotic plaques in the relevant blood vessels. As the reduction in the size of the plaque is demonstrated, it can be extrapolated to these diseases. With the use of the described preparation, it is feasible to achieve laudable balance of different lipids.

The present invention is further explained in the form of following exemplary illustrations. However, it is to be understood that the example is merely an illustrative and are not to be taken as limitations upon the scope of the invention. Various changes and modifications to the disclosed embodiments will be apparent to those skilled in the art. Such changes and modifications are made without departing from the scope of the invention.

The herbal formulation prepared using the above mentioned composition and process is used for treating any kind of animal including mammals that can benefit from the methods of treatment include but not limited to apes, chimpanzees, orangutans, humans, monkeys; and domesticated animals such as dogs, cats, horses, cattle, pigs, sheep, goats, chickens, mice, rats, guinea pigs, and hamsters. The tests were done on the selected animal by grouping them into plurality of groups preferably four include intact animals, control animals, untreated animals under myocardial infarction simulation, treated animals receiving the herbal formulation with 5 animals in each group. Preferably the experiment was made on wistar male rats.

The preparation of myocardium tissue specimens for the histological test was performed on an automatic processor with a parafing embedding. The slices of 3-5 microns were stained with hematoxiling and eosin, van gieson's and weigert'spicro-fuchsin. The microscopic analysis for analyzing the test results was performed on a microscope and the image analysis was performed in the software application.

For analyzing the count of red blood cells and leukocyte formula is performed on a hematology analyzer adapted for veterinarian research. Form the biochemical blood tests comprises the determination of the activity of enzymes in the blood plasma such as alanine aminotransferase (ALT, 2.6.1.2), aspartate aminotransferase (AST, 2.6.1.1), creatine phosphokinase (CPK. 2.7.3.2.) [symbol] lactate dehydrogenase (LDH 1, 2, 1.1.1.27). All biochemical tests were conducted using the available standard reagent kits. The statistical analysis is performed by using a software application for verifying statistical hypotheses the level of confidence used.

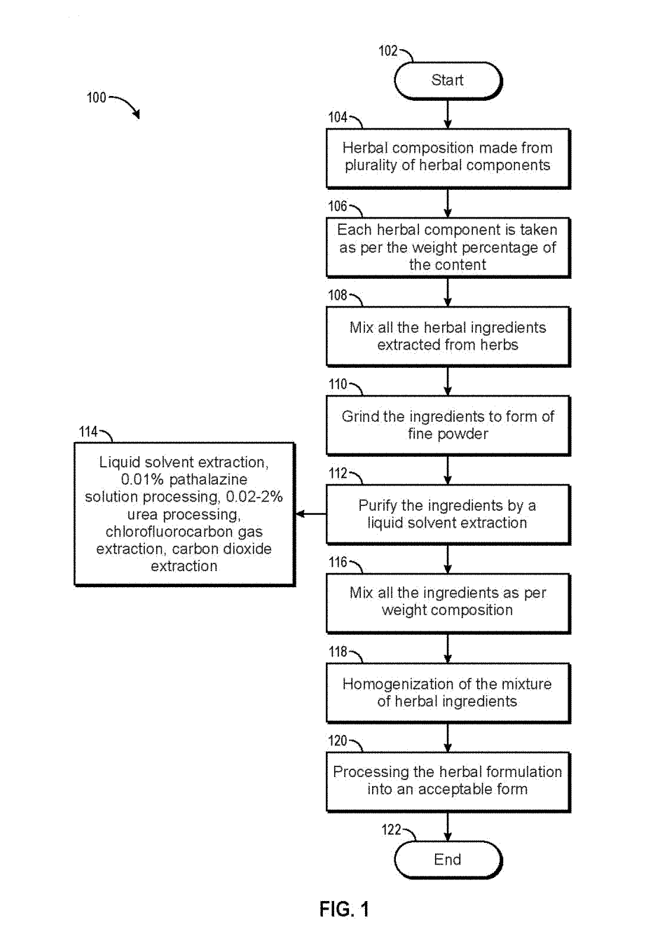

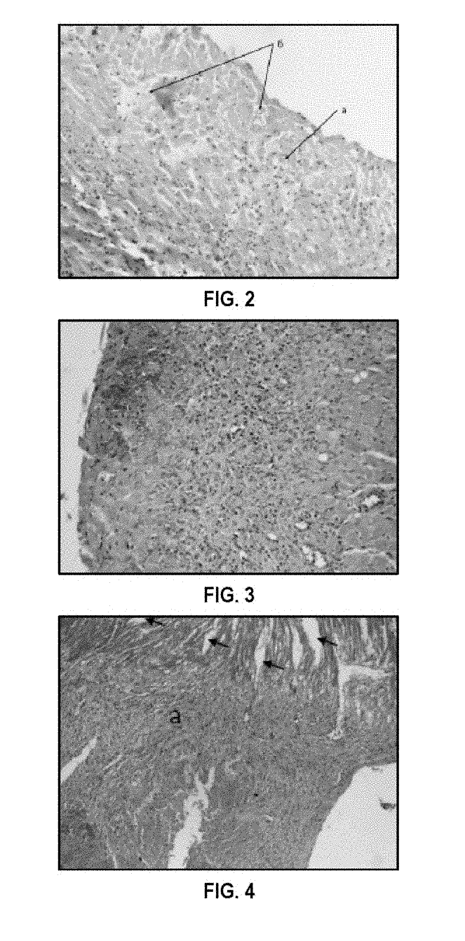

The historical examination of the heart of the animal in the 1^{st} day after the experimental simulation of the myocardial infarction localizing the infarction area with transmural subepicardial distribution is shown in the FIG. 2. An apparent exudative inflammatory reaction is observed in response to the injury. The area of necrosis shows the diffuse infiltration of polymorphonuclear leukocytes (PMN). The alterative processes in the myocardium are manifested by the necrosis of cardiomyocytes, the phenomena of karyolysis, plasmolysis, plasmorrhexis. In the perifocal area of the infarctionthere was detected a myomalacia as a part of cardiomyocytes with the collapse of the sarcoplasmic structures and the apparent interstitial edema. Plethora was determined in the endomysium vessels with the phenomenon of adiemorrhysis and focal eritrodiapedesis.

On the 7th day of the experiment, the first granulations in the perifocal area of infarction are formed surrounded by macrophages and fibroblasts. At this stage moderately apparent signs of exudative inflammation continue along with the presence of migration of PMNs, which carry the phagocytic function i.e. phagocyte and lyse necrotic mass as shown in the FIG. 3.

On the 14th day of the experiment, the infarcted area was localized predominantly subcpicardially without transmural distribution as shown in the FIG. 4. In the perifocal area cardiomyocytes were detected with signs of degradation, exhibited by the lysis of sarcoplasm, pyknosis of the nuclei of cardiomyocytes. The emerging granulation tissue in the perifocal area was represented by loose connective tissue with a large amount of functionally active fibroblasts. At this stage, the granulations were infiltrated with lymphoid elements without PMNs present.

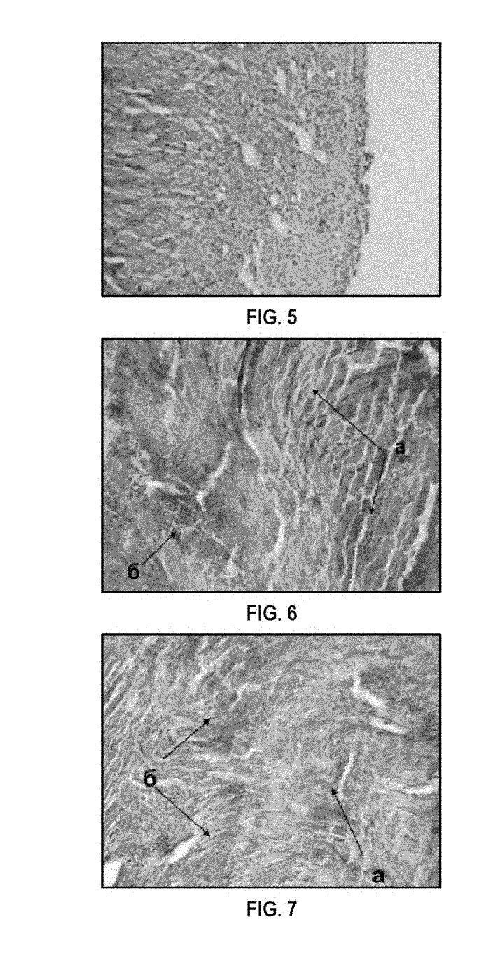

FIG. 5 illustrates the infraction area on the 14^{th} day without administering the herbal formulation identifies the destructive changes of cardiomyocytes in the area bordering to myocardial infraction. This also emerges granulation tissue infiltrated by lymphoid elements.

One day after the operation performed on the animal treated with herbal formulation a portion of muscle tissue necrosis was detected in the left ventricle spreading transmurally up to the half of the ventricular wall as shown in FIGS. 6&7. The necrotic cardiomyocytes with hemorrhage portions were located along the periphery of the defect. The leukocyte infiltration was observed, extending over the endomysium inside the myocardium. There is no sign of the formation of leukocytal circumvallate separating the necrotic tissue are detected.

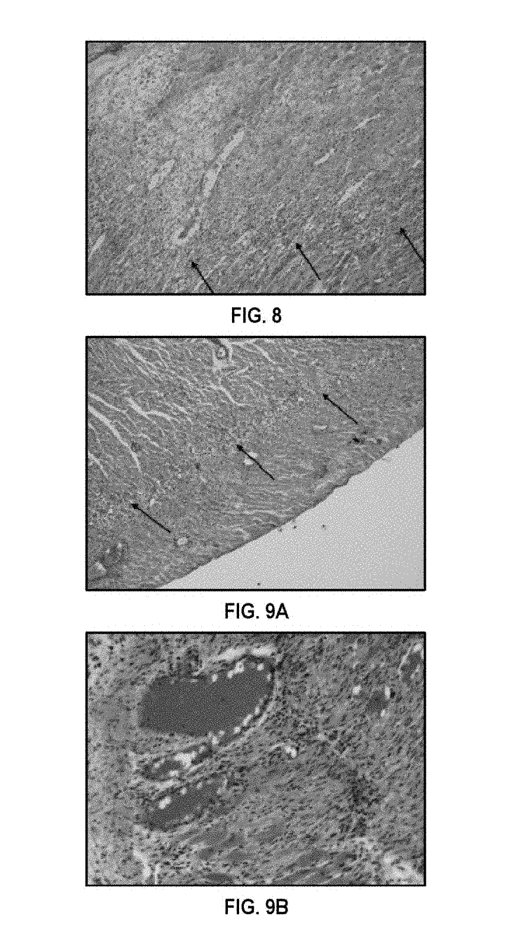

As illustrated in the FIG. 8, the images shows the 5^{th} effect of the area of necrosis in the treated animals was subepicardially localized, limited from the intact myocardium by the formed leukocytal circumvallate. At the bottom of the defected signs of the formation of granulation tissue were detected and the intensity of leukocyte infiltration decreases is also shown in FIG. 8.

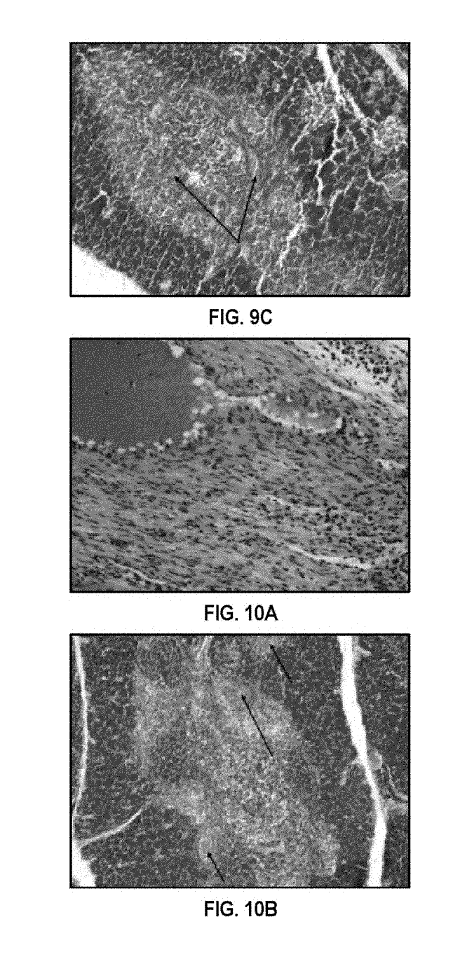

FIG. 9a illustrates a 7^{th} day of forming the subepicardial area of myocardial necrosis and the granulation tissue, in which there are macrophages, proliferating fibroblasts that form scar tissue fibrous structures. Sinusoidal capillaries are formed in the loose connective tissue.

As shown in FIGS. 9a & 9b, infraction area and thymus on the 14th and 1st day of the operation with herbal formulation notified the reaction of the thymus on the infarction simulation, which manifested itself in the plethora of vessels of the medullar substance with the formation of sludge complexes in them. Meanwhile, the structure of the organ is preserved with clear boundaries of cortex and medulla by the 7^{th} day the histological structure of the thymus corresponds to normal.

The infraction area on the 14th day after the operation with herbal formulation is shown in FIG. 10a, describing the presence of connective tissue in the area of necrosis, presented by the collagen and elastic fibers, capillary type vessels. In the intercellular substance cellular component is reduced. The regulatory cells of lymphoid series are also found. After the treatment with the herbal formulation, thickening of the pericardium is observed in the area of experimental infarction due to proliferating fibroblasts and the formation of collagen structures. Subepicardial vessels are dilated and plethoric. Deteriorated cardiomyocytes are replaced with elements of the connective tissue. There are no PMNs found in the area of scar formation. The infiltration of scar tissue with small amounts of lymphocytes and macrophages is observed.

As shown in the FIG. 10b illustrating the thymus on the 5^{th} day after operating with the herbal formulation showing the plethora of the vessels of medullar substance. The infarcted area is represented by a scar with the predominance of the cellular component. The scar appears to have a significant number of hemosiderophages, macrophages, lymphocytes, plasma cells.

The experimental tests conducted on any animal using the herbal formulation of the present invention depicting in the experimental simulation of infarction has shown the reduction in the exudative infiltration phase in the necrosis area, quick reoccurrence of the phase of productive-proliferative inflammation, which contributes to the early development of granulation tissue and the formation of connective tissue scar.

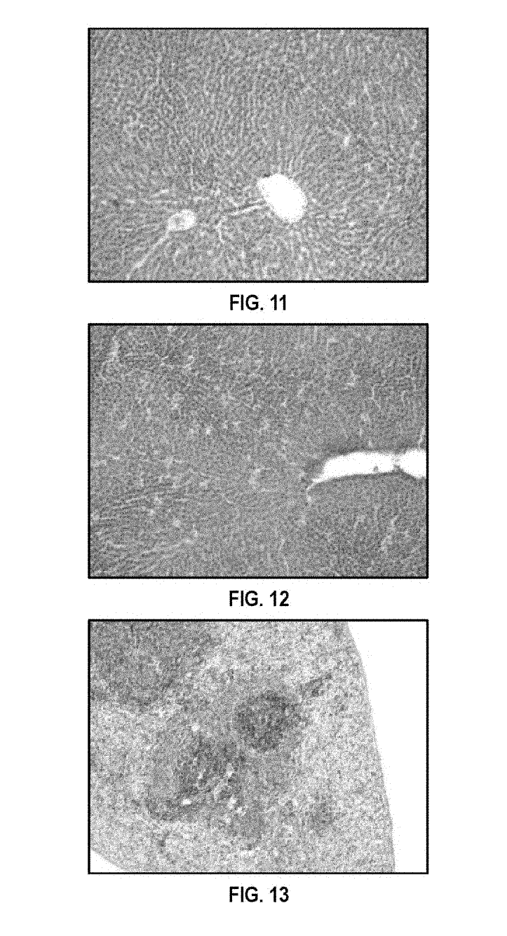

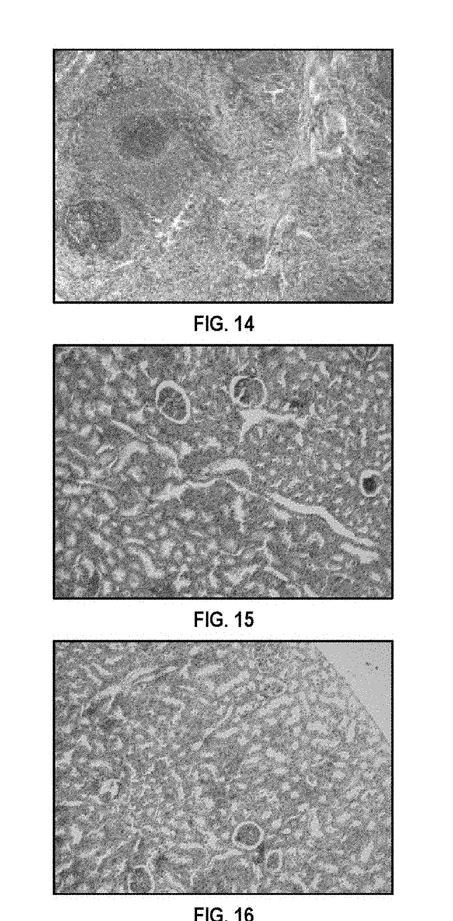

The examination of immunopoiesis organs (thymus and spleen), as well as liver and kidney in the course of the infarction simulation and treatment with herbal formulation is shown in the FIGS. 11-16 under the administration shows no pathological changes found in these organs.

The test results were shown in the plurality of tables by treating the animal with herbal composition of the present invention as described below. Animals were randomized to treatment groups in the order of their pre-determined randomized number. Following tables illustrates the results of tests conducted on animals.

TABLE 1
Indices of peripheral blood

| | | | Red blood | | |
|---|---|---|---|---|---|
| | | | cells, | | |
| | | Hemoglobin, | millions | | Blood platelets, |
| | | grams per | per | | thousands per |
| Groups | | decaliter | microliter | Hematocrit, % | microliter |
| Intact animals | | 14.4 ± 0.5 | 8.95 ± 0.11 | 44.3 ± 0.8 | 731.1 ± 45.0 |
| Control animal (preparation | | 16.0 ± 0.6 | 9.23 ± 0.13 | 44.4 ± 0.8 | 608.0 ± 35.0 |
| under the invention) | | | | | |
| Myocardialin | 14.2 ± 1.0 | 8.05 ± 0.79 | 38.2 ± 3.1 | 672.6 ± 54.4 | 672.6 ± 54.4 |
| farction | 13.9 ± 0.7 | 9.02 ± 0.14 | 39.2 ± 2.9 | 684 ± 50.2 | 684 ± 50.2 |
| | 13.5 ± 0.6 | 7.94 ± 0.51 | 39.0 ± 2.1 | 715.7 ± 50.9 | 715.7 ± 50.9 |
| | 14.3 ± 0.6 | 9.02 ± 0.44 | 40.5 ± 2.8 | 728.0 ± 48.2 | 728.0 ± 48.2 |
| Myocardialin | 15.9 ± 0.4 | 9.33 ± 0.14 | 44.3 ± 0.9 | 588.5 ± 40.5 | 588.5 ± 40.5 |
| farction + | 14.8 ± 0.2 | 8.76 ± 0.28 | 41.5 ± 1.0 | 523.0 ± 87.4 | 523.0 ± 87.4 |
| preparation under | 14.2 ± 0.5 | 8.85 ± 0.48 | 42.6 ± 2.4 | 692.9 ± 40.3 | 692.9 ± 40.3 |
| the invention | 14.2 ± 0.6 | 8.92 ± 0.24 | 43.6 ± 2.7 | 710.7 ± 42.7 | 710.7 ± 42.7 |

Based on the test results shown in the above table showed no significant abnormalities in the count of hemoglobin, erythrocytes, hematocrit value and the number of platelets neither after the simulation of myocardial infarction, nor after the preventive administration of herbal preparation to animals, followed by the myocardial infarction simulation. The red blood parameters, platelets and hematocrit are not sufficiently informative for the disease. The absence of significant changes in these parameters in animals receiving only the herbo-mineral preparation (control animals) as compared with the intact rats indicates the absence of adverse effects of repeated administration of herbal preparation. The used herbo-mineral preparation did not cause the occurrence of disorders such as anemia, or any change in one of the main indicators of hemostasis that the platelet count can be considered.

TABLE 2
Leucocytes of the peripheric blood

| | | Leucocytes, | Lymphocytes | | Mean cells | | Granulocytes | |
|---|---|---|---|---|---|---|---|---|
| | | thousands | thousands | | thousands | | thousands | |
| | | per | per | | per | | per | |
| Groups | | microliter | microliter | % | microliter | % | microliter | % |
| Intact animals | | 8.5 ± 0.5 | 4.4 ± 0.3 | 51.7 ± 3.8 | 0.9 ± 0.3 | 11.3 ± 1.3 | 3.2 ± 0.5 | 37.0 ± 3.8 |
| Control | | 8.8 ± 0.5 | 4.4 ± 0.7 | 48.7 ± 5.4 | 1.2 ± 0.3 | 14.0 ± 1.6 | 3.2 ± 0.4 | 37.3 ± 5.0 |
| animals (preparation | | | | | | | | |
| under the invention) | | | | | | | | |
| Myocardial | Day 1 | 10.5 ± 1.0 | 5.8 ± 0.5 | 55.8 ± 2.8 | 0.8 ± 0.4 | 8.2 ± 3.4 | 3.9 ± 0.8 | 36.0 ± 5.5 |
| Infarction | Day 5 | 13.9 ± 0.8* | 5.9 ± 0.7 | 43.6 ± 4.4 | 0.8 ± 0.4 | 7.9 ± 3.0 | 7.2 ± 0.8* | 48.5 ± 4.8* |
| | Day 7 | 14.0 ± 0.7* | 5.4 ± 0.4 | 38.3 ± 2.0 | 0.9 ± 0.4 | 6.3 ± 2.7 | 7.8 ± 0.9* | 55.3 ± 4.7* |
| | Day 14 | 11.3 ± 1.6 | 4.8 ± 0.6 | 44.5 ± 4.2 | 0.9 ± 0.4 | 7.0 ± 2.8 | 5.6 ± 0.8* | 48.5 ± 5.2 |
| Myocardial | Day 1 | 7.4 ± 2.5 | 2.9 ± 0.8** | 40.0 ± 3.0* ** | 0.4 ± 0.2 | 4.5 ± 1.6* | 4.2 ± 1.5 | 55.5 ± 1.5* ** |
| infarction + | Day 5 | 10.9 ± 1.6 | 5.2 ± 1.2 | 46.7 ± 3.7 | 0.8 ± 0.4 | 7.0 ± 3.2 | 4.9 ± 0.3* ** | 46.3 ± 6.5 |
| preparation | Day 7 | 11.1 ± 1.9 | 5.4 ± 0.6 | 47.5 ± 4.2 | 0.9 ± 0.3 | 8.3 ± 3.3 | 4.8 ± 0.4* ** | 44.2 ± 4.2 |
| under the | Day 14 | 10.7 ± 1.2 | 4.8 ± 0.5 | 44.9 ± 4.3 | 0.8 ± 0.3 | 8.6 ± 2.9 | 5.1 ± 0.8 | 46.5 ± 4.7 |
| invention | | | | | | | | |

*The difference with the group of intact animals is reliable if P < 0.05.
**The difference of the treated animals with the control animals (myocardial infarction) is reliable if P < 0.05.

In the above table the total count of leukocytes and their fractions are examined. There observed no changes regarding white blood indices in the control rats. Therefore the herbal formulation caused no phenomena of inflammation or imbalance in the composition of individual fractions of leukocytes. After simulating the myocardial infarction there discovered an increase in the total count of leukocytes on the 5th-7th day after the operation approximately 1.6 times above that of intact animals as shown in the Table 2. Moderate leukocytosis in rats with myocardial infarction developed due to the fraction of granulocytes, an increase in the count of which was recorded when measured both in absolute units and as a percentage ratio.

The hypoxia and collapse of cardiomyocytes in the infarction area increase the inflammatory response, leading to the generation of inflammatory mediators, which cause the appearance in the necrosis area of activated neutrophils that belong to the fraction of granulocytes, with the consequent neutrophil-mediated cytoxic damage. The activated neutrophils and free radicals produced by them bring about the endothelial dysfunction and the secondary damage of cardiomyocytes. The amount of neutrophils in the peripheric blood is considered to be the diagnostic and prognostic index, characterizing the dimension of the infarcted area in the myocardium.

The administration of preventive peroral of the herbal formulation was helpful in decreasing the inflammatory process after the myocardial infarction simulation, since no significant change of the total number of leucocytes was detected in this group, and the increase of the absolute quantity of granulocytes in the group of treated animals was significantly lowered in the untreated rats as shown in Table 2.

From tables 3-7 presented below depicts the results of determination of enzyme activity in blood plasma in the control group of animals that received after determining with herbal formulation. The indices of activity of all the examined enzymes of blood plasma (AST, ALT, CPK, aggregate activity, activity of isoenzymes LDH) remained at the level of indices measured in the intact animals. Therefore the mixture of herbs do not cause any adverse effects on the myocardium, liver and other organs, which could be the source of these enzymes in the event of adverse action in the research.

TABLE 3
Activity of aspartate aminotransferase (AST), U/l (micromol/l * min)

| | Control animal | | | | |
|---|---|---|---|---|---|
| Intact | (preparation under | | | | |
| animals | the invention) | Day 1 | Day 5 | Day 7 | Day 14 |
| | | Myocardial infarction | | | |
| 11.6 ± 0.8 | 11.4 ± 0.9 | 24.9 ± 2.0* | 17.4 ± 2.0* | 17.3 ± 1.4* | 17.2 ± 0.4* |

| Myocardial infarction + preparation under the invention | | | |
|---|---|---|---|
| 23.8 ± 2.6* | 12.6 ± 0.7 | 13.3 ± 0.8** | 13.2 ± 1.1** |

*The difference with the group of intact animals is reliable if P < 0.05.
**The difference of the treated animals with the control animals (myocardial infarction) is reliable if P < 0.05.

After simulating the myocardial infarction without the preliminary effect of the herbal composition, there was noticed an increase in activity of all the examined enzymes in the blood plasma, and this increase depended on the time that had elapsed after the operation. For instance, the activity of AST and ALT increased almost two times on the 1st day of the experiment and remained increased almost 1.5 times as of the 5th, 7th and 14th day as compared to that of the control rats as shown in Tables 3 and 4.

TABLE 4
Activity of alanine aminotransferase (ALT), U/l (micromol/l * min)

| | Control animals | | | | |
|---|---|---|---|---|---|
| Intact | (preparation under | | | | |
| animals | the invention) | Day 1 | Day 5 | Day 7 | Day 14 |
| | | Myocardial infarction | | | |
| 10.5 ± 0.7 | 10.6 ± 0.5 | 23.7 ± 3.7* | 18.7 ± 3.3* | 17.5 ± 1.6* | 15.3 ± 2.2 |

| Myocardial infarction + preparation | | | |
|---|---|---|---|
| under the invention | | | |
| 16.1 ± 0.7* | 8.7 ± 0.2** | 10.8 ± 0.8** | 10.2 ± 1.2 |

*The difference with the group of intact animals is reliable if P < 0.05.
**The difference of the treated animals with the control animals (myocardial infarction) is reliable if P < 0.05.

TABLE 5
Aggregate activity of creatine phosphokinase (CPK), U/l (micromol/l * min)

| | Control | | | | |
|---|---|---|---|---|---|
| | animals (preparation | | | | |
| Intact | under the | | | | |
| animals | invention) | Day 1 | Day 5 | Day 7 | Day 14 |
| | | Myocardial infarction | | | |
| 910.2 ± 63.2 | 866.7 ± 148.9 | 1607.1 ± 270.6* | 1646.3 ± 3.0* | 1681.8 ± 285.6* | 927.5 ± 153.7 |

| Myocardial infarction + preparation under the invention | | | |
|---|---|---|---|
| 1559.4 ± 51.7* | 1626.3 ± 139.2* | 1684.3 ± 102.5* | 1024.4 ± 121.1 |

*The difference with the group of intact animals is reliable if P < 0.05.

As depicted in the table 5, there is an increase in the aggregate activity of CPK almost 1.5 times the index of the intact group on 1^{st}-7^{th} day of experiment with normalization by the 14^{th} day. A similar pattern in rats with experimental myocardial infarction was established for the aggregate activity of LDH wherein the source of various isoenzymes can be myocardium, liver and skeletal muscles. The exceeding above the norm by 1.8 times on the 1^{st}-7^{th} day of experiment and return to the index level of control rats by the 14^{th} day.

TABLE 6
Aggregate activity of lactate dehydrogenase (LDH), U/l (micromol/l * min)

| | Control animals | | | | |
|---|---|---|---|---|---|
| Intact | (preparation under | | | | |
| animals | the invention) | Day 1 | Day 5 | Day 7 | Day 14 |
| | | Myocardial infarction | | | |
| 256.0 ± 39.5 | 236.4 ± 50.5 | 477.6 ± 4.7* | 470.3 ± 25.2* | 462.5 ± 27.7* | 259.6 ± 20.0 |

| Myocardial infarction + preparation under the invention | | | |
|---|---|---|---|
| 463.9 ± 40.5* | 279.9 ± 34.0** | 290.7 ± 29.0** | 260.4 ± 34.4 |

*The difference with the group of intact animals is reliable if P < 0.05.
**The difference of the treated animals with the control animals (myocardial infarction) is reliable if P < 0.05.

TABLE 7
Activity of isoensyms of lactate-dehydrogenase 1,2 (LDH1,2), U/l (micromol/l * min)

| | | | | | |
|---|---|---|---|---|---|
| | Control animals | | | | |
| | (preparation | | | | |
| Intact | under the | | | | |
| animals | invention) | Day 1 | Day 5 | Day 7 | Day 14 |

| Myocardial infarction | | | |
|---|---|---|---|
| 450.2 ± 4.6* | 262.3 ± 22.1* | 346.5 ± 53.0* | 155.3 ± 71.3 |

| | | |
|---|---|---|
| 146.4 ± 23.5 | 121.4 ± 30.3 | Myocardial infarction + preparation under the invention |

| | | | |
|---|---|---|---|
| 288.4 ± 21.3*** | 135.2 ± 52.7 | 140.3 ± 28.9** | 141.4 ± 33.3 |

*The difference with the group of intact animals is reliable if P < 0.05;
**The difference of the treated animals with the control animals (myocardial infarction) is reliable if P < 0.05.

There was also found a significant increase inactivity of LDH 1, 2 isoenzymes specific for the myocardium in blood plasma as shown in the Table 7. The deviation from the norm of this index by the 1^{st} day after the operation was the highest compared with the deviation from the norm of other enzymes by the 14^{th} days of activity LDH1.2 also normalized.

The simulation of myocardial infarction in rats that preventively received the herbal formulation accompanied by a less apparent cytolytic syndrome. The normalization of aminotransferases as shown in Tables 3 and 4, aggregate activity of LDH as shown in Table 6 and isoensymes of LDH as shown in Table 7 came about faster in treated animals compared to the untreated ones. However, the dynamics of CPK do not undergo any significant changes under the effect of the herbal formulation as shown in Table 5. Therefore, the preventive repeated administration of the claimed formulation contributed to the decrease of inflammatory process and cytolysis specific to the experimental myocardial infarction.

In contrast, a series of tests and experiments were conducted by excluding even a single component from the claimed formulation which is depicting a significant negative influence on the indices examined. Therefore, the composition of the preparation is a unique formulation in reducing the after-effects of the myocardial infarction. As for the other designations of the claimed formulation, the possibility of realization of these designations stems from the mentioned data of the experiment and the epiopathogenesis of the processes.

For example, the materials demonstratively prove the possibility of using the claimed formulation for reducing atherosclerotic plaques, as a lipid lowering agent or as an anti-inflammatory agent as well as for treating stroke and ischemia. The cardioprotective properties of the claimed formulation cannot be disputed after getting acquainted with the mentioned data and same is true for the other mentioned designations. In support to the above fact that the achieved effect is a result is the combination of all the components of the herbal formulation, certain data is presented below.

TABLE 8
Indices of the peripheric blood

| | | Hemoglobin | | | |
|---|---|---|---|---|---|
| | | grams | Red blood | | Blood platelets, |
| | | per | cells millions | | thousands per |
| Groups | | decaliter | per microliter | Hematocrit, % | microliter |
| Intact animals | | 14.4 ± 0.5 | 8.95 ± 0.11 | 44.3 ± 0.8 | 731.3 ± 45.0 |
| Control animals | | 14.9 ± 0.6 | 9.0 ± 0.13 | 44.4 ± 0.8 | 680.0 ± 35.0 |
| under the invention, with the | | | | | |
| exclusion of the pearlshell | | | | | |
| powder) | | | | | |
| Myocardial Infarction | Day 1 | 14.2 ± 1.0 | 8.05 ± 0.79 | 38.2 ± 3.1 | 672.6 ± 54.4 |
| | Day 5 | 13.9 ± 0.7 | 9.02 ± 0.14 | 39.2 ± 2.9 | 684 ± 50.2 |
| | Day 7 | 13.5 ± 0.6 | 7.94 ± 0.51 | 39.0 ± 2.1 | 715.7 ± 50.9 |
| | Day 14 | 14.3 ± 0.6 | 9.02 ± 0.44 | 40.5 ± 2.8 | 728.0 ± 48.2 |
| Myocardial | Day 1 | 15.0 ± 0.4 | 8.9 ± 0.14 | 44.2 ± 0.9 | 608.5 ± 40.5 |
| infarction + | Day 5 | 14.2 ± 0.2 | 8.6 ± 0.28 | 41.0 ± 1.0 | 599.0 ± 83.4 |
| preparation under the | Day 7 | 14.2 ± 0.5 | 8.85 ± 0.48 | 42.6 ± 2.4 | 692.9 ± 40.3 |
| invention, with the | Day 14 | 14.2 ± 0.6 | 8.9 ± 0.24 | 43.5 ± 2.7 | 719.7 ± 42.7 |
| exclusion of the pearl | | | | | |
| shell powder | | | | | |

The above table shows that only by the 7th day the blood counts came to correspond the indices when the formulation used as a whole.

TABLE 9
Leucocytes of the peripheric blood

| | | Leucocytes, | Lymphocytes | | Mean cells | | Granulocytes | |
|---|---|---|---|---|---|---|---|---|
| | | thousands | thousands | | thousands | | thousands | |
| | | per | per | | per | | per | |
| Groups | | microliter | microliter | % | microliter | % | microliter | % |
| Intact animals | | 8.5 ± 0.5 | 4.4 ± 0.3 | 51.7 ± 3.8 | 0.9 ± 0.3 | 11.3 ± 1.3 | 3.2 ± 0.5 | 37.0 ± 3.8 |
| Control | | 8.7 ± 0.5 | 4.4 ± 0.7 | 48.7 ± 5 | 1.2 ± 0.3 | 14.0 ± 1 | 3.2 ± 0.4 | 37.3 ± 5.0 |
| animals (preparation | | | | | | | | |
| under the | | | | | | | | |
| invention without | | | | | | | | |
| Swarnabhasma) | | | | | | | | |
| Myocardial | Day 1 | 10.5 ± 1.0 | 5.8 ± 0.5 | 55.8 ± 2.8 | 0.8 ± 0.4 | 8.2 ± 3.4 | 3.9 ± 0.8 | 36.0 ± 5.5 |
| infarction | Day 5 | 13.9 ± 0.8* | 5.9 ± 0.7 | 43.6 ± 4.4 | 0.8 ± 0.4 | 7.9 ± 3.0 | 7.2 ± 0.8* | 48.5 ± 4.8* |
| | Day 7 | 14.0 ± 0.7* | 5.4 ± 0.4 | 38.3 ± 2.0 | 0.9 ± 0.4 | 6.3 ± 2.7 | 7.8 ± 0.9* | 55.3 ± 4.7* |
| | Day 14 | 11.3 ± 1.6 | 4.8 ± 0.6 | 44.5 ± 4.2 | 0.9 ± 0.4 | 7.0 ± 2.8 | 5.6 ± 0.8* | 48.5 ± 5.2 |
| Myocardial | Day 1 | 9.0 ± 2.5 | 4 ± 0.8** | 50.0 ± 3.0* ** | 0.6 ± 0.2 | 6.1 ± 1.6* | 4.1 ± 1.5 | 40.5 ± 1.5* ** |
| infarction + | Day 5 | 12.0 ± 1.6 | 5.2 ± 1.2 | 46.7 ± 3 | 0.8 ± 0.4 | 7.0 ± 3.2 | 4.9 ± 0.3* ** | 46.3 ± 6.5 |
| preparation | Day 7 | 13.1 ± 1.9 | 5.4 ± 0.6 | 47.5 ± 4.4 | 8.9 ± 0.3 | 8.3 ± 3.3 | 4.8 ± 0.4* ** | 44.2 ± 4.2 |
| under the | Day 14 | 11.0 ± 1.2 | 4.8 ± 0.5 | 44.9 ± 4.1 | 0.8 ± 0.3 | 8.6 ± 2.7 | 5.2 ± 0.8 | 46.5 ± 4.7 |
| invention | | | | | | | | |
| without | | | | | | | | |
| Swarnabhasma | | | | | | | | |

*The difference with the group of intact animals is reliable if P < 0.05.
**The difference of the treated animals with the control animals (myocardial infarction) is reliable if P < 0.05.

The above table shows that the indices come to normal only by the 5^{th} day when the formulation under the invention is used.

TABLE 10
Activity of aspartate aminotransferase (AST), U/l (micromol/l * min)

| | Control animals | | | | |
|---|---|---|---|---|---|
| | (preparation under | | | | |
| Intact | the invention | | | | |
| animals | without Triphala) | Day 1 | Day 5 | Day 7 | Day 14 |
| | | Myocardial infarction | | | |
| 11.6 ± 0.8 | 11.5 ± 0.9 | 24.9 ± 2.0* | 17.4 ± 2.0* | 17.3 ± 1.4* | 17.2 ± 0.4* |

| Myocardial infarction + preparation under the invention | | | |
|---|---|---|---|
| without Triphala | | | |
| 24.4 ± 2.6* | 13.9 ± 0.7 | 13.7 ± 0.8** | 13.3 ± 1.1** |

*The difference with the group of intact animals is reliable if P < 0.05.
**The difference of the treated animals with the control animals (myocardial infarction) is reliable if P < 0.05.

The above table has demonstrated the effect of protection of cardiomyocytes and regeneration through the examples of infarction in rats.

It will be appreciated by those of ordinary skill in the art that changes could be made to the embodiments made above without departing from the broad inventive concept thereof. It can be understood, therefore, that this invention is not limited to the particular embodiments disclosed, but is intended to cover modifications within the spirit and scope of the present invention as defined by the appended claims. Additionally, the process and aspects incorporated into one embodiment discussed above are understood to be incorporated into all other embodiments unless otherwise stated.

1. A herbal formulation for reducing the after effects of myocardial infarction to protect cardiomyocytes which ultimately shortens the risk of disease and its impact, the composition essentially comprising of:
a terminalia arjuna in an amount of 2-90% by weight of the composition;
a pashanbhedi more specifically a bergenia lingulata in an amount of 2-90% by weight of the composition;
ajatamansimore specifically a nardostachys jatamansi in an amount of 2-90% by weight of the composition;
aswamabhasmamore specifically a gold ultra fine powder in an amount of 2 ppm-6 ppm by weight of the composition;
agandhakrasayan in an amount of 2 ppm-6 ppm by weight of the composition;
atrifalamore specifically a three myrobalans in an amount of 2-90% by weight of the composition;
acommiphoramukul in an amount of 2-90% by weight of the composition; and
a calcined pearl shell powder more specifically a mukthapisthi in an amount of 1.25-10% by weight of the composition.

2. The herbal formulation according to claim 1, wherein the composition more specifically comprises of:
the said terminalia arjuna in an amount of 33.3% by weight of the composition;
the said pashanbhedi more specifically a bergenia lingulata in an amount of 16.7% by weight of the composition;
the said jatamansi more specifically a nardostachys jatamansi in an amount of 10% by weight of the composition;
the said swamabhasma more specifically a gold ultra fine powder in an amount of 6 ppm by weight of the composition;
the said gandhakrasayan in an amount of 2 ppm by weight of the composition;
the said trifala more specifically a three myrobalans in an amount of 23.3% by weight of the composition;
the said commiphoramukul in an amount of 15.4% by weight of the composition; and
the said calcined pearl shell powder more specifically a mukthapisthi in an amount of 1.25% by weight of the composition.

3. The herbal formulation according to claim 1, wherein the composition is prepared in at least one form selected from a group include:
a gelatin capsule;
a vegetarian capsule;
a tablet;
a liquid;
a syrup;
a dry beverage; and
a snack bar.

4. The herbal formulation according to claim 1, wherein the composition can also be used in various forms comprising an atherosclerotic plaque reducer, as an anti-oxidant, as an anti-inflammatory agent, as an anxiolytic, as a cardioprotective agent, as a cardiac tonic for the treatment of cardiac related problems includes:
myocardial infarction;
stroke;
ischemia;
intermittent claudication;
post-MI scar; and
for the regeneration of tissue destroyed as a result of MI.

5. The herbal formulation according to claim 1, wherein the composition consisting of various components includes a mixture of active ingredients extracted from the herbs.

6. The herbal formulation according to claim 1, wherein brings about improvement in the serum lipid fractions and results in improvement of ratios of these fractions.

7. The herbal formulation according to claim 1, wherein facilitates blood thinning properties by modulating thrombocyte action leading to dis-aggregation of thrombocytes.

8. The herbal formulation according to claim 1, wherein leverages Mesenchymal Stem cell migration of CD 133, CD 117 and CD 90 to the site of myocardial tissue injury leading to total regeneration of the heart tissue in a post MI scenario.

9. A method for preparing a herbal formulation for reducing the after effects of myocardial infarction to protect cardiomyocytes which ultimately shortens the risk of disease and its impact, the method comprises steps of:
grinding the herbal ingredients forming a formulation to form a fine powder;
purifying the herbal ingredients through a plurality of ways to prepare an effective formulation;
homogenization of herbal ingredients to attain a homogenous preparation;
mixing the herbal ingredients according to the weight of the composition of the components; and
processing the mixed formulation into an acceptable form of administration.

10. The method of purifying the herbal ingredients according to claim 6, characterized to include one or more methods include:
a liquid solvent extraction;
a 0.01% phthalazine solution processing followed by extraction;
a 0.02-2% urea processing followed by extraction;
a chlorofluorocarbon gas extraction; and
a carbon dioxide extraction.

11. The method according to claim 6, characterized for processing the herbal mixture into an acceptable form include but not limited to:
a gelatin capsule;
a vegetarian capsule;
a tablet;
a liquid;
a syrup;
a dairy beverage; and
a snack bar.

12. The method according to claim 6, characterized to prepare a herbal formulation of the composition can also be used in various forms comprising an atherosclerotic plaque reducer, as an anti-oxidant, as an anti-inflammatory agent, as an anxiolytic, as a cardioprotective agent, as a cardiac tonic for the treatment of cardiac related problems include:
myocardial infarction;
stroke;
ischemia;
intermittent claudication;
post-MI scar; and
for the regeneration of tissue destroyed as a result of MI.
